(19) United States
(12) Patent Application Publication
(10) Pub. No.: US 2004/0058056 A1
(43) Pub. Date: Mar. 25, 2004

(54) Drug diffusion coatings, applications and methods

(76) Inventors: Shigemasa Osaki, Sandy, UT (US); Paul O. Zamora, Gaithersburg, MD (US); Meng Chen, Silverspring, MD (US)

Correspondence Address: PEACOCK MYERS AND ADAMS P C, P O BOX 26927, ALBUQUERQUE, NM, 871256927

(21) Appl. No.: 10/332,307

(22) PCT Filed: Jul. 6, 2001
(86) PCT No.: PCT/US01/41281

(51) Int. Cl. A61L 2/00

(52) U.S. Cl. 427/2.1; 427/489; 427/497; 427/578

(57) ABSTRACT

Methods, coatings and coated medical devices are provided, wherein a plasma-deposited aliphatic polymerized hydrocyclosiloxane membrane is deposited as a diffusion control barrier for a drug deliver component consisting of one or more therapeutic agents coated on a surface or contained within a matrix, preferably a polymeric matrix, coated on a surface. The plasma-polymerized hydrocyclosiloxane membrane coats all or substantially all of the drug delivery component in contact or communication with the exterior surface, such that all or substantially all of any drug or therapeutic agent must diffuse across the membrane in order to be released.

### CROSS-REFERENCE TO RELATED APPLICATIONS

This application claims the benefit of the filing of U.S. Provisional Patent Application Serial No. 60/216,915, entitled Plasma Polymerized Siloxane Membrane As Diffusion Control Barrier, filed on Jul. 6, 2000 and the specification thereof is incorporated herein by reference.

### BACKGROUND OF THE INVENTION

1. Field of the Invention (Technical Field)

The invention relates to compositions and methods for coating implantable medical device surfaces to provide controlled release of a therapeutic agent. In one embodiment, the invention relates to a polymeric composition applied to at least one surface of the device, the composition including a polymeric matrix and the therapeutic agent, wherein the therapeutic agent is a drug, peptide, biological agent or other bioactive molecule, which polymeric composition has applied thereto by plasma deposition a hydrocyclosiloxane-containing membrane.

2. Background Art

Note that the following discussion refers to a number of publications by authors and year of publication, and that due to recent publication dates certain publications are not to be considered as prior art vis-a-vis the present invention. Discussion of such publications herein is given for more complete background and is not to be construed as an admission that such publications are prior art for patentability determination purposes.

The use of plasma polymerized siloxane membranes and coatings are known in the art, and is taught in U.S. Pat. No. 5,463,010, Hydrocyclosiloxane Membrane Prepared by Plasma Polymerization Process, incorporated herein by reference. These siloxane membranes provide a variety of benefits, and can be used to coat substrates to impart properties such as hydrophobicity, thromboresistance, gas permeability and biocompatability.

It is also known that drugs or other therapeutic agents may be coated on a surface, or may alternatively comprise a part of a porous or degradable matrix, such that the drugs or other therapeutic agents are eluted over a period of time. However, in prior art methods the rate of elution is controlled by the design of the matrix, design of the binding elements contained in the coating material, or the like. As a result any change in the rate of elution requires, in most instances, a reformulation of the coating material, reformulation of the matrix material, or the like, with attendant testing and evaluation.

U.S. Pat. Nos. 5,624,411; 5,679,400; and 5,464,650 disclose a method and device for delivery of a drug using an intraluminal stent, consisting of a first coating layer that includes a therapeutic substance and a second coating layer that includes a porous polymer. In one embodiment, the first coating layer also includes a porous polymer, which may be the same or different as the polymer of the second coat. However, in all embodiments the polymer is porous and is applied by art conventional means, such as by application of a solution and optionally a solvent.

U.S. Pat. Nos. 6,096,070; 5,824,049; and 5,609,629 disclose application of a bioactive substance layer on a medical device, but without any polymeric matrix, with subsequent deposition of a porous polymer over the bioactive substance layer. In a preferred method, the porous polymer is deposited by vapor deposition, and is from about 500 to about 25,000 nm thickness, and is optimally about 5,000 nm thick. The polymeric layer is characterized in that it has defined pores. Use of plasma deposition of a polymer of tetramethyldisiloxane is suggested therein, but polymers thereof, such as polydimethylsiloxane, are known to comprise a simple homogenous monolayer which permits passage of drug molecules in thicknesses as much as 0.12 mm. Barry, B. W., 1983, Dermatological Formulations: Percutaneous Absorption. Marcel Dekker, New York; Pefile, S. C. et al: Int. J. Pharmaceutics 161 (1998) 237-243.

U.S. Pat. Nos. 5,569,463 and 5,447,724 disclose a method of drug release including a polymeric reservoir containing an elutable drug, with a surface layer that contains defined pores, such as a polyether urethane composition.

These and other patents disclose a number of methods and devices for controlled release of drugs or other bioactive substances. However, none of the prior art methods meet all the requirements for the intended purpose, including a variable quantity of drug, a variable release rate, minimal thickness of the membrane for controlled release, and high strength and hardness of the topmost membrane, all in a biocompatible and thromboresistant material. There is thus a need for a coating or membrane material with desirable properties for use in medical devices, and which is biocompatible and thromboresistant, and which also can be employed to control the rate of release of drugs or other therapeutic agents which are coated on a surface or within a matrix.

### SUMMARY OF THE INVENTION (DISCLOSURE OF THE INVENTION)

In one embodiment, the invention provides a biocompatible coating composition for in vivo diffusion of a therapeutic agent, which composition includes a layer including a therapeutic agent dispersed in a polymeric matrix and a membrane posited over the layer, the membrane formed from the plasma polymerization of hydrocyclosiloxane monomer of the general formula:

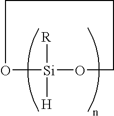

where R is an aliphatic group having 1 to about 5 carbon atoms and n is an integer from 2 to about 10, wherein the membrane cross-links with the polymeric matrix of the layer.

In a related embodiment, the biocompatible coating composition includes a layer containing a therapeutic agent, with the membrane as set forth above posited over the layer. In this embodiment, no polymeric matrix is provided.

The invention further provides an implantable medical device, which medical device includes a structural component adapted for implantation is a patient, the structural component having at least one exterior surface. A layer including a therapeutic agent is posited over at least a portion of the at least one exterior surface, and a membrane posited over the layer, the membrane formed from the plasma polymerization of hydrocyclosiloxane monomer of the general formula:

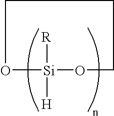

where R is an aliphatic group having 1 to about 5 carbon atoms and n is an integer from 2 to about 10. The implantable medical device further includes embodiments wherein the layer comprising a therapeutic agent includes a polymeric matrix, whereby the therapeutic agent is dispersed in the polymeric matrix and the membrane cross-links with the polymeric matrix.

The invention further provides a method of applying a biocompatible coating composition to a structural component for in vivo diffusion of a therapeutic agent, the method including the steps of providing a structural component adapted for introduction into a patient, the structural component having at least one exterior surface; positing a layer including a therapeutic agent over at least a portion of the at least one exterior surface; and plasma depositing a membrane over the layer including the therapeutic agent, the membrane formed from the plasma polymerization of hydrocyclosiloxane monomer of the general formula:

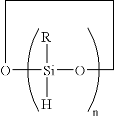

where R is an aliphatic group having 1 to about 5 carbon atoms and n is an integer from 2 to about 10. In this method, the step of positing the layer comprising a therapeutic agent may further consist of positing a layer including a polymeric matrix and the therapeutic agent.

In each of the foregoing embodiments, n may be between 7 to 10, between 4 to 6 or between 2 to 3. The hydrocyclosiloxane monomer may be 1,3,5,7-tetramethylhydrocyclotetrasiloxane, 1,3,5,7,9-pentamethylhydrocyclopentasiloxane, 1,3,5,7,9,11-hexamethylhydrocyclohexasiloxane, or a mixture of 1,3,5,7,9-pentamethylcyclopentasiloxane and 1,3,5,6,9,11-hexamethylcyclohexasiloxane monomers.

The polymeric matrix, if provided, may include poly(2-hydroxyethyl methacrylate), polycaprolactone or cellulose acetate butyrate.

In each of the foregoing embodiments, the membrane has a thickness of between about 10 nm and about 450 nm, and preferably between about 20 and about 250 nm. The layer including the therapeutic agent contains between about 0.01 mg and about 5.0 mg of therapeutic agent per cm², and preferably between about 0.1 mg and about 0.5 mg of therapeutic agent per cm².

A primary object of the present invention is to provide a method and device for drug diffusion for use with implantable medical devices.

Another object of the present invention is to provide a method and device for drug diffusion wherein the rate of diffusion is controlled by a plasma-deposited hydrocyclosiloxane membrane that is from about 20 to about 450 nm, and preferably about 20 to about 250 nm, in thickness.

Another object of the invention is to provide a polymeric matrix through which a therapeutic drug is dispersed, which polymeric matrix has a first rate of diffusion, with a plasma-deposited hydrocyclosiloxane membrane posited thereover, and preferably cross-linked with the polymeric matrix, most preferably highly cross-linked, the plasma-deposited hydrocyclosiloxane membrane having a second rate of diffusion.

A primary advantage of the present invention is that it provides a very hard and thin membrane, with a thickness of from about 20 to about 450 nm thickness, and preferably about 20 to about 250 nm thickness, that controls the rate of diffusion.

Another advantage of the present invention is that plasma-deposited hydrocyclosiloxane forms a highly cross-linked and dense membrane, which membrane is also hard and flexible, but not elastic, such that it provides surface protection, is highly biocompatible, and controls diffusion.

Other objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 is a plot illustrating the thickness of a hydrocyclosiloxane coating of this invention;

FIG. 2 is plot illustrating the rate of elution of NPC-15199 as a function of hydrocyclosiloxane coating time; and

FIG. 3 is a plot illustrating the rate of elution of daunomycin as a function of hydrocyclosiloxane coating time.

### DESCRIPTION OF THE PREFERRED EMBODIMENTS

### BEST MODES FOR CARRYING OUT THE INVENTION

The present invention provides methods and coatings for the formation and use of a plasma-deposited aliphatic polymerized hydrocyclosiloxane membrane as a diffusion control barrier for drugs and therapeutic agents coated on a surface or contained within a matrix, preferably a polymeric matrix. The drugs or therapeutic agents, together with the membrane and, if provided, the matrix, form the drug delivery component of the invention. In use, the plasma-polymerized hydrocyclosiloxane membrane coats all or substantially all of the drug delivery component that is in contact or communication with the exterior surface, whereby all or substantially all of any drug or therapeutic agent must transit the membrane in order to be released. The membrane has a variety of characteristics in addition to serving as a diffusion control membrane, including thromboresistance, gas permeability and biocompatibility. The present invention is particular useful with medical devices.

The membrane is formed through plasma polymerization of suitable aliphatic hydrocyclosiloxane monomers or plasma copolymerization of aliphatic hydrocyclosiloxane monomers and co-monomers, depending on the desired characteristics. Aliphatic hydrocyclosiloxane monomers have the general formula:

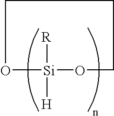

wherein R is alkyl group of 1 to about 5 carbon atoms and n is an integer from 2 to about 10. Monomers include those where n is 7 to 10, where n is 4 to 6 and where n is 2 to 3. Co-monomers such as fluorocarbons, organo-based monomers, or functional group terminated monomers can be utilized to change the properties of the membrane to adjust for varied applications.

Definitions. For purposes of this patent, the following terms are defined:

The term “biocompatible polymer” refers to polymers which, in the amounts employed, are not toxic and are substantially non-immunogenic when placed internally in the patient.

The term “bioabsorbable polymer” refers to biocompatible polymers that are degradable, and preferably biodegradable, with a definable degradation rate. In general, a bioabsorbable polymer is capable of being broken down, in the body, into smaller constituents. Preferably the bioabsorbable polymer is, as it degrades into smaller constituents, metabolized or excreted through normal biological systems. Hydrolysis is one mechanism by which some bioabsorbable materials are broken down following implantation within a living organism. Some bioabsorbable polymers may be composites, and may have a bioabsorption rate that varies over time. Examples of suitable bioabsorbable polymers may include poly-L-lactide, poly-D-lactide, polyglycolide, poly(dioxanone), polycaprolactone, polygluconate, polylactic acid-polyethylene oxide copolymers, modified cellulose, collagen, glycosaminogylcans including hyluronic acid and cross-linked hyaluronic acid, fibrin, elastin, silk, poly(hydroxybutyrate), polyanhydride, polyphosphoester, poly(amino acids), poly(alphahydroxy acid) and combinations thereof.

The term “plasma polymerization” refers to the formation of polymeric materials under the influence of plasma, consisting of ionized gases, free radicals and electrons.

The term “plasma glow zone” refers to the region in which the glow discharge in the plasma polymerization process takes place.

The term “therapeutic agent” refers to any substance used as a drug, to effect a biochemical change in an organism, or to confer a benefit to an organism. The term thus includes art conventional drugs, compounds, molecules, peptides, peptidomimetics, antibodies and fragments and mimics thereof, and the like. A therapeutic drug may, but need not, bind to a receptor in the organism, be a receptor for an endogenous substance found in an organism, or be an agonist, antagonist, or a mixed agonist-antagonist of a receptor-mediated process or reaction.

Structural Components. The coatings may be applied to any of a wide variety of structural components of medical devices. Suitable structural components with a surface include medical devices that are intended to contact blood or other tissues, such as a stent, catheter, shunt, graft, artificial blood vessel, nerve-growth guide, artificial heart valve, prosthetic, pacemaker lead, indwelling catheter, cardiovascular graft, bone replacement, wound healing device, cartilage replacement device, urinary tract replacement and other medical devices known in the art. Other examples of medical devices that would benefit from the application of the present invention will be readily apparent to those skilled in the art of surgical and medical procedures and are therefore contemplated by the instant invention. The structural component may include a mesh, coil, wire, inflatable balloon, or any other device or structure which is capable of being implanted at a target location, including intravascular target locations, intralumenal target locations, target locations within solid tissue, such as for the treatment of tumors, and the like. The implantable device can be intended for permanent or temporary implantation. Such devices may be delivered by or incorporated into intravascular and other medical catheters.

Suitable surfaces of the structural component include stainless steel, nitinol, titanium, other metal alloys, polyvinyl chloride, polyethylene, polylactide, poly glycolide, poly caprolactone, poly methyl methacrylate, poly hydroxylethyl methacrylate, polyurethane, polystyrene, polycarbonate, dacron, extended poly tetrafluoroethylene (Teflon®), related fluoropolymer composites (Gore-Tex®), or combinations thereof. All or part of the available surface can be modified. Other substrate materials can also be used, including poly(acrylate), poly(bisphenol A carbonate), polybutadiene, poly(butylene terephthalate), poly(butryl methacrylate), polydimethylsiloxane, polyester, polyethyleneimine, polysulfone, poly(vinyl acetate), polyvinylidine fluoride, polylactide, polyglycolide, polycaprolactone and copolymers and variants thereof.

In one embodiment, the structural component may be a biodegradable or bioerodible material, which after controlled release of a therapeutic drug degrades or erodes. The use of biodegradable or bioerodible materials to provide sustained or controlled release of chemotherapeutic or other drugs, including bioactive drugs, has been known for a number of years. Biodegradable implants for the controlled release of hormones, such as contraceptive hormones, were developed over twenty years ago, and have been used as birth control devices. Biodegradable or bioerodible materials employed for controlled release of drugs include polyanhydrides, polyglycolic acid, polylactic/polyglycolic acid copolymers, polyhydroxybutyrate-valerate and other aliphatic polyesters, among a wide variety of polymeric substrates employed for this purpose. Biodegradable implantable materials, some of which have been used in drug delivery systems, are described in U.S. Pat. Nos. 5,656,297; 5,543,158; 5,484,584; 4,897,268; 4,883,666; 4,832,686; and 3,976,071. U.S. Pat. No. 5,876,452 describes biodegradable polymeric material, such as polyanhydrides and aliphatic polyesters, providing substantially continuous release of bioactive drugs, including bi-phasic release of bioactive drugs. In one embodiment, a bioabsorbable polymeric structural component is made from a biocompatible polymeric material such as polycaprolactone, poly(D,L-lactide) poly(L-lactide), polyglycolide, poly(dioxanone), poly(glycolide-co-trimethylene carbonate), poly(L-lactide-co-glycolide), poly(D,L-lactide-co-glycolide), poly(L-lactide-co-D,L-lactide) or poly(glycolide-co-trimethylene carbonate-co-dioxanone). In one embodiment, the persistence of the bioabsorbable polymeric structural component within a living organism is in excess of the anticipated period over which the therapeutic agent will diffuse in an effective amount, and preferably in excess of at least two such anticipated periods.

Therapeutic Agents. Any of a variety of drugs or therapeutic agents may be employed as a part of the drug delivery component of the device. The drug delivery component includes any drug suitable for treatment of the disease condition for which the device is employed. For cancer and similar neoplastic diseases, this includes any known chemotherapeutic agent, including but not limited to bleomycin, busulfan, carboplatin, carmustine, cisplatin, dactinomycin, daunorubicin, doxorubicin, estramustine, interferon, levamisole, methotrexate, mitomycin, paclitaxel, pentostatin, plicamycin, tamoxifen, vinblastine, vindesine and the like. This also includes radiosensitizers including 5-halo-uracils, anti-angiogenesis compounds including thalidomide and tranilast, natural or synthetic peptide hormones including octreotide, and compounds that induce apoptosis including butyrate and nitric oxide donors. Any drugs or therapeutic agents can be used singly or in combination.

The therapeutic agent used in the present invention can be virtually any therapeutic agent that possesses desirable therapeutic characteristics for application to a tissue. Non-limiting classes of useful bioactive agents of the present invention include antithrombogenic agents, antibiotic agents, anti-tumor agents, antioxidants, antimetabolite agents, antiviral agents, anti-angiogenic agents, angiogenic agents, anti-mitotic agents, anti-inflammatory agents, angiostatin agents, endostatin agents, cell cycle regulation agents, bioactive peptides, peptide mimetics, protein fragments, genetic agents, including hormones, such as estrogen; and homologs, analogs, derivatives, fragments, pharmaceutical salts and mixtures of any of the foregoing.

The therapeutic agent includes both solid substances and liquid substances. Anti-thrombogenic agents include, for example, glucocorticoids (e.g. dexamethasone, betamethasone), hirudin, tocopherol, coumadin, angiopeptin, aspirin, ACE inhibitors and dipyridamole. Anti-mitotic agents and antimetabolite agents include drugs such as methotrexate, azathioprine, vincristine, vinblastine, fluorouracil, doxorubicin, daunomycin, taxanes including paclitaxel, rapamycin, and mutamycin.

Moreover, the therapeutic agent of the present invention can include organic acid functional group-containing antibiotics. Such antibiotics include rifampicin, penicillins, cephalosporins, vancomycins, aminoglycosides, quinolones, polymyxins, erythromycins, tetracyclines, chloramphenicols, clindamycins, lincomycins, and sulfonamides, and homologs, analogs, fragments, derivatives, pharmaceutical salts and mixtures of any of the foregoing.

The therapeutic agent of the present invention can also include organic acid functional group-containing anti-tumor agents. Such anti-tumor agents include paclitaxel, docetaxel, alkylating agents including mechlorethamine, chlorambucil, cyclophosphamide, melphalan and ifosfarnide; antimetabolites including methotrexate, 6-mercaptopurine, 5-fluorouracil and cytarabine; plant alkaloids including colchicines, vinblastine, vincristine and etoposide; antibiotics including doxorubicin, daunomycin, bleomycin, and mitomycin; nitrosureas including carmustine and lomustine; inorganic ions including cisplatin; hormones including somatostatin, LHRH, progesterone, and estrogen; steroid hormones including hydrocortisone, tamoxifen, and flutamide; and homologs, analogs, fragments, derivatives, pharmaceutical salts and mixtures of any of the foregoing.

The therapeutic agent of the present invention can also include organic acid functional group-containing anti-viral agents. Such anti-viral agents include amantadines, rimantadines, ribavirins, idoxuridines, vidarabines, trifluridines, acyclovirs, ganciclovirs, zidovudines and foscarnets, and homologs, analogs, fragments, derivatives, pharmaceutical salts and mixtures of any of the foregoing.

Polymeric Matrix. The coating includes the therapeutic drug which is dispersed within and throughout a polymeric matrix, preferably a matrix formed from a biocompatible polymer, and in one embodiment a matrix formed from a bioabsorbable polymer.

Bioabsorbable polymers that may be employed include poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. In addition, biostable polymers with a relatively low chronic tissue response such as polyurethanes, silicones, and polyesters can be employed, and other polymers can also be employed, provided they can be dissolved in the selected solvent and cured or polymerized on the surface of the structural component such as polyolefins, polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other; olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose. The polymeric matrix material can, of course, be made from other polymers depending upon the factors set forth herein. Such a choice of polymeric matrix materials is within the knowledge of those skilled in the art.

In a preferred embodiment, poly(2-hydroxyethyl methacrylate) (“PHEMA”) is utilized as the polymeric matrix material. PHEMA is soluble in a mixture of alcohol and water, such as 60% or 80% ethanol and in a mixture of acetone and water. This solvent system can also be employed with many therapeutic agents, and is thus a preferred polymeric matrix material, particularly for therapeutic agents soluble in ethanol and water, or in acetone and water.

Other preferred polymeric matrix materials include polycaprolactone and cellulose acetate butyrate, both of which are soluble in solvents compatible with many therapeutic agents.

The polymeric matrix material is preferably soluble in a solvent in which the therapeutic agent is also soluble. Such solvents include, without limitation, water, alcohols, chloroform, acetone, xylene, and the like. The polymeric matrix material, therapeutic agent and solvent must be compatible, such that there is no adverse chemical change in the therapeutic agent affecting efficacy of the therapeutic agent.

It is preferred that the therapeutic agent also be soluble in the solvent, such that both the polymeric matrix material and the therapeutic agent are both in solution. However, in an alternative embodiment, the therapeutic agent may be dispersed through the solution of the polymeric matrix material and the solvent. In such event, the therapeutic material is most preferably in the form of fine particulates.

The solution comprising a solvent, the therapeutic agent and the polymeric matrix material may be applied to the structural component by any means known in the art. This can include dip coating, painting, spraying, partial immersion and the like. Following application, the coated structural component may be incubated at a constant temperature, such as between about 37° C. and about 80° C., and preferably between about 45° C. and 65° C., to facilitate evaporation of the solvent and curing of the polymeric matrix. Multiple coats of the solution may be applied to the structural component, optionally with curing between applications. In this way, the thickness of the coating, and the quantity of therapeutic agent per unit area, may be precisely controlled.

The quantity of polymeric matrix material placed in solution depends, in part, on the solubility of the polymeric matrix material. Thus with, for example, PHEMA in an ethanol and water solvent, between about 5% and 20% PHEMA, preferably between about 7% and 10% PHEMA, may be placed in solution. With other polymeric matrix materials the maximum and optimal concentration in the selected solvent may be easily and empirically ascertained.

The quantity of the therapeutic agent per unit area is dependent, in part, on the solubility of the therapeutic agent in the solvent, the quantity of therapeutic agent either in solution or dispersed therein, the thickness of the coating and the number of coats applied. The coating, including all applications thereof, may thus contain between about 0.01 mg per cm² and about 5.0 mg per cm² of therapeutic agent, and preferably between about 0.1 mg per cm² and about 0.5 mg per cm² of therapeutic agent.

The ratio of therapeutic drug to polymeric matrix material depends, in large part, on the polymer selected, the desired rate of release of the therapeutic drug, and the like. This parameter may be altered as required to obtain a desired result.

In a preferred embodiment, the polymeric matrix material forms a cross-linked polymer, and accordingly contains appropriate reactive groups that may be cross-linked. Thus, PHEMA, for example, contains hydrogen atoms along its carbon backbone, and may thus be cross-linked to both itself and to a plasma-deposited hydrocyclosiloxane membrane that contains Si—H groups. A cross-linkable polymeric matrix material is preferred for several reasons, including increased adherence to the structural component, and of more significance, forming a cross-linked connection with the plasma-deposited hydrocyclosiloxane membrane. Cross-linking between the polymeric matrix material and the plasma-deposited hydrocyclosiloxane membrane is believed to more precisely control the rate of diffusion, increase adherence of the membrane to the coating, and define a harder and more protective membrane.

The polymeric matrix material may also cross-link with the surface of the structural component. Thus, the structural component may be selected such that cross-linking is possible, or may be modified to enhance cross-linking. Such modifications include, but are not limited to, introduction of amine groups such as by plasma etching with NH₃ or other nitrogen-containing gases.

The coating including the polymeric matrix may optionally have a predefined release rate as a result of the coating composition, which may be a continuous, bi-phasic or an otherwise modulated release rate. The therapeutic drug is locally released at the site of the device, and is cleared from the patient by normal clearance and excretory function. The drug and other components, including a polymeric matrix, is selected such that it may be coated with a hydrocyclosiloxane membrane by plasma polymerization.

The device may itself consist solely of a polymeric matrix and the drug or other therapeutic agent, and preferably a biodegradable polymeric matrix, which is then coated with a hydrocyclosiloxane membrane by plasma polymerization, wherein the length of plasma polymerization determines the thickness of the siloxane membrane, and accordingly the rate of diffusion of the drug or other therapeutic agent. In all cases, however, the polymeric matrix will remain substantially intact for a predetermined persistence period, such persistence period being at least equal to the maximum length of time of drug diffusion.

Hydrocyclosiloxane Monomer Plasma Polymerization. The hydrocyclosiloxane monomers are polymerized directly on the drug delivery component surface using plasma-state polymerization techniques. The general process of plasma-state polymerization is known to those in the art. See Yasuda, Plasma Polymerization, Academic Press Inc., New York (1985), incorporated herein by reference.

In brief, hydrocyclosiloxane monomers are polymerized onto a surface by activating the monomer from a gaseous state in a plasma state composed of electrons, ions, gas atoms, free radicals and molecules. The plasma state generates highly reactive species of the hydrocyclosiloxane monomer, which forms a characteristically highly cross-linked, ultra-thin polymer membrane, which is deposited on the substrate surface as it moves through the area of most intense energy density, the plasma glow zone.

In practice, an electric discharge from a radio frequency (R.F.) generator is applied to the “hot” electrodes of plasma reactor. The selected monomers are introduced into the reactor and energized into a plasma, saturating the plasma glow zone with an abundance of energetic free radicals and lesser amounts of ions and free electrons produced by the monomers. As material including the drug delivery component passes through or remains in the plasma glow zone, the surface of the material is continually bombarded with free radicals, resulting in the polymerized membrane coating. The plasma-state polymerized hydrocyclosiloxane membrane is highly adherent to most organic and inorganic materials, providing a smooth and hard membrane coating.

When the plasma glow zone is activated, the monomer or monomer mixture is continually passed through the plasma glow zone. The material to be coated, such as the structural component to which is adhered a therapeutic drug, and preferably a therapeutic drug in a polymeric matrix, is placed within the plasma glow zone. This results in a flow of plasma state monomer or monomer mixture in and around the structural component, thereby resulting in deposition on exposed surfaces. The monomer or monomer mixture that does not deposit is removed under vacuum from the plasma field. The plasma state monomer or monomer mixture deposition may be controlled by varying the plasma conditions, including primarily the power level of the R.F. generator and the length of time the target material is in the plasma glow zone, typically the total length of time of plasma generation.

Aliphatic hydrocyclosiloxane monomers may be used to create a homogeneous membrane coating. Alternatively, aliphatic hydrocyclosiloxane monomers and co-monomers may be mixed to create membrane coatings having properties different from the properties of a homogenous membrane prepared using aliphatic hydrocyclosiloxane monomers. For example, by introducing reactive functionalizing monomers, or organo-based monomers, or fluorocarbon monomers together with the aliphatic hydrocyclosiloxane monomers in the plasma polymerization system, chemical affinity of the plasma copolymerized aliphatic hydrocyclosiloxane membrane with selective monomers can be controlled. This allows use of the copolymerized plasma membrane for applications that require the membrane to differentiate between certain types of gases, ions, and molecules.

By controlling the mole ratio of the functionalizing monomers, the chemical structure and physical properties of the siloxane copolymer plasma polymerized membrane may be systematically changed. This allows for variable properties of the membrane as a diffusion membrane, with a different diffusion rate per unit thickness of the membrane.

The following four different types of plasma polymerized aliphatic hydrocyclosiloxane membranes (Types A-D) represent useful embodiments of the invention.

“Type A” refers to membrane coatings that are deposited on the substrate surface through the plasma state polymerization process using aliphatic hydrocyclosiloxane monomers of the general formula:

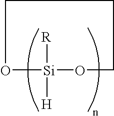

where R is an aliphatic group and n is an integer from 2 to about 10, preferably 4 to 6. Preferred aliphatic hydrocyclosiloxane monomers include: 1,3,5,7-tetramethylcyclotetrasiloxane (“TMCTS”); 1,3,5,7,9-pentamethylhydrocyclopentasiloxane (“PMCTS”); 1,3,5,7,9,11-hexamethylhydrocyclohexasiloxane (“HMCHS”) and a mixture of 1,3,5,7,9-pentamethylcyclopentasiloxane and 1,3,5,6,9,11-hexamethylcyclohexasiloxane monomers (“XMCXS”). Use of a radio frequency power greater than 5 Watts (“W”), a system pressure less than 300 mTorr, and a monomer flow rate greater than about 1 standard cm³ per minute (“sccm”), will cause a homogeneous, hard, hydrophobic, biocompatible, gas permeable membrane to form on the surface passing through the plasma glow zone.

“Type B” refers to membrane coatings that are produced by plasma co-polymerization process of mixtures of the same aliphatic hydrocyclosiloxane monomers used in Type A membrane coatings and fluorocarbon monomers. Suitable fluorocarbon monomers would include CF₄, C₂F₆, C₃F₆, C₃F₈, C₂F₄, hexafluoropropene, perfluorobenzene, ditrifluoromethylbenzene, perfluoro-2-butyltetrahydrofuran, and pentafluorostyrene. The linear alkyl-type fluorocarbon monomers should have C/F ratio greater than ¼, for example, C₃F₆. If the C/F ratio is below ¼, etching usually occurs in the plasma polymerization process.

“Type C” refers to membrane coatings which are produced by plasma co-polymerization process of mixtures of the same aliphatic hydrocyclosiloxane monomers used in Type A membrane coatings and organo-based monomers. Suitable organo-based monomers would include ethylene, allylamine, and N-trimethylsilylallylamine, hydrocarbons, unsaturated amines (both N-protected and N-unprotected), cyclic aliphatic amines (both N-protected and N-unprotected), mercaptans (organosulfur), nitrites and organophosphorous compounds.

“Type D” refers to membrane coatings that are produced by plasma co-polymerization process of mixtures of the same aliphatic hydrocyclosiloxane monomers used in Type A membrane coatings and reactive functionalizing monomers. Suitable functionalizing monomers include N₂, CO₂, NH₃ and SO₂.

The thickness of the membrane can be controlled precisely during the plasma polymerization process, and in general the thickness of the membrane coating is a direct function of the length of time of plasma polymerization, assuming a constant flow rate of monomer. Thus the thickness of the membrane may be controlled by the length of time of plasma polymerization. The diffusion rate of the drug or therapeutic agent through the membrane is, in turn, related to the specific composition of the membrane and the thickness of the membrane. In a preferred embodiment, the thickness of the membrane is no more than about 450 nm, and is preferably from about 20 nm to about 250 nm in thickness, depending on the rate of diffusion desired with the specific drug or therapeutic agent, and the specific composition of the membrane.

The plasma polymerization process parameters may be varied, so long as a polymer with the desired characteristics is obtained. For example, the RF generator power may be varied from about 5 W to about 200 W or higher, depending on the desired rate of plasma deposition, the configuration of the plasma apparatus and the like. Similarly, the mass flow rate of the monomer or monomer mixture may be altered as desired. In general, the mass flow rate setting and the R.F. power setting are synchronized, such that there is sufficient monomer available to polymerize, without requiring excessive removal of unutilized material.

The molecular size, configuration, net charge, polarity and solubility of the therapeutic agent also affect the rate of diffusion. Thus, as is shown in the Examples, one therapeutic agent may diffuse at one rate through a plasma-polymerized hydrocyclosiloxane membrane of a given thickness, while another therapeutic agent diffuses through a membrane of the same thickness at a different rate. The diffusion characteristics for any given therapeutic agent may be easily determined by empirical means, as may the thickness of membrane required to result in diffusion at the desired rate. Since the rate of diffusion is proportional to thickness, only minimal empirical data is required to specify the thickness required for a desired rate of diffusion.

The hydrocyclosiloxanes of this invention, and particularly TMCTS, are characterized in part by a high density of cross-linked sidechains when polymerized, specifically when polymerized by plasma deposition. This is believed to result in a very high-density membrane, wherein the density of molecular packing with the cross-linked polysiloxanes is high. This is believed to be related, in part, to the cyclic nature of hydrocyclosiloxanes, as opposed to linear siloxanes, which form a simple homogenous monolayer. The polymerized hydrocyclosiloxanes are further characterized by the presence of Si—H groups, which presence may be conveniently detected by use of infrared (“IR”) spectroscopy. Other siloxane polymers, such as polydimethylsiloxane, do not contain Si—H groups detectable by IR spectroscopy, even though such groups may be present in the monomer prior to polymerization. The Si—H groups in plasma-polymerized hydrocyclosiloxanes are believed to contribute to cross-linking, both within the polymer and between the polymer and any substrate, such as a therapeutic agent or a polymeric matrix containing a therapeutic agent.

Thus plasma polymerized hydrocyclosiloxanes are characterized by forming, under the plasma deposition conditions disclosed hereunder, a hard yet elastic, highly cross-linked and dense membrane, which is not significantly elastic. Further, the plasma-polymerized hydrocyclosiloxanes of this invention are only minimally soluble in most solvents, including water.

It is known that the density of a diffusion medium and the solubility of the diffusant in the diffusion medium are parameters that control the diffusion flux, including the rate of diffusion. Prior art plasma-deposited siloxanes have employed polydimethylsiloxanes, which are comparatively significantly less dense, less cross-linked and substantially more soluble than the polymers of this invention. The use of the polymers of this invention thus provides much harder and denser membranes, which will control diffusion at a rate comparable to that obtainable with polymer described in the prior art, such as polydimethylsiloxane, but at a thickness of between about one-tenth and about one-one hundredth or less than that required for polydimethylsiloxane. Thus, the membranes of this invention can be 20 nm in thickness or thinner, and still provide a significant decrease in diffusion of a therapeutic agent posited thereunder. Similarly, the maximum thickness required for a membrane of this invention is between about 250 and 450 nm in thickness. A membrane of this invention that is substantially thicker is relative impermeable, such that therapeutic agents will not diffuse across such membrane in meaningful quantities or rates.

In one alternative embodiment, the diffusion control barrier siloxane membrane may be applied to a device that consists of the drug delivery component. In such instance, the drug delivery component may be an implantable structure forming the device, which may be a biodegradable structure.

In an alternative embodiment, the therapeutic agent may be dissolved in a solvent, and directly applied to the structural component without use of a polymeric matrix material. More than one coat of the therapeutic agent may be applied, optionally with curing by incubation as for the polymeric matrix material. A plasma-deposited hydrocyclosiloxane membrane is then applied over the therapeutic agent, with the composition of the membrane and thickness of the membrane controlled so as to obtain the desired rate of diffusion, and thus rate of release of the therapeutic agent. Preferably, the plasma-deposited hydrocyclosiloxane cross-links with the topmost portions of the therapeutic agent in this embodiment, and the therapeutic agent is selected such that it may be cross-linked.

Industrial Applicability:

The invention is further illustrated by the following non-limiting examples.

### EXAMPLE 1

Table 1 summarizes the time of plasma deposition using TMCTS and the thickness of the resulting membrane as determined using atomic force microscopy (AFM) following plasma deposition of TMCTS on a silicone substrate. The plasma was generated at 83 W and 55 mTorr with a mass flow rate of 84 sccm.

TABLE 1

| TMCTS Deposition | | Standard |
|---|---|---|
| time (minutes) | Thickness (nm) | Deviation |
| 0.66 | 10.7 | 2.9 |
| 1 | 18.5 | 1.8 |
| 2 | 64.4 | 1.05 |
| 4 | 118.7 | 1.02 |
| 6 | 135.5 | 3.51 |

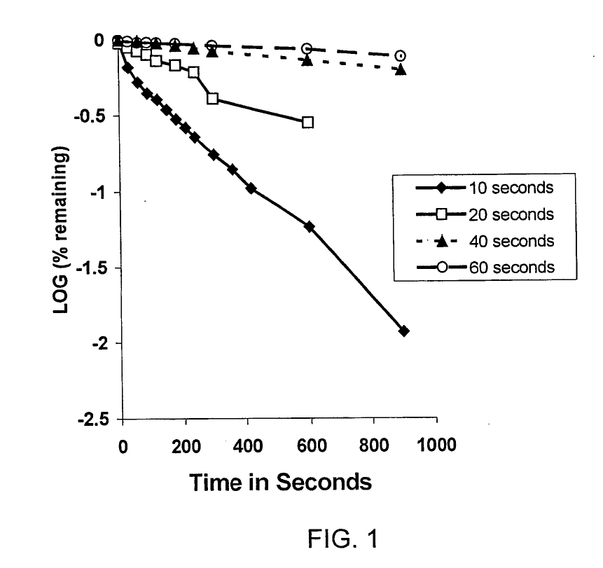

FIG. 1 graphically depicts the data of the foregoing table, illustrating the thickness of TMCTS on a silicon wafer as a function of plasma deposition time.

### EXAMPLE 2

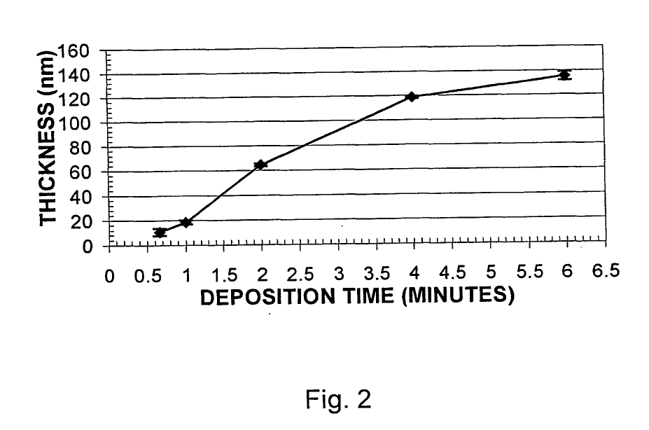

N-(9-fluorenylmethoxycarbonyl)-L-leucine (NPC-15199), an anti-inflammatory drug, was coated on a stainless steel coupon by dip-coating the coupon in a mixture of 20 mM NPC-15199 in a 60:40 solution of acetone:water containing 7.5% poly(2-hydroxyethyl methacrylate) (“PHEMA”). The coupon was then air dried for about 5 minutes at approximately 60° C. TMCTS plasma was deposited on different NPC-15199 coated coupons for 20, 40, 60 and 80 seconds, respectively. The coated coupons were then a buffered saline solution at pH 7.4 at room temperature. Elution of NPC-15199 was measured by change in absorbance using a spectrophotometer. The increase of the half-elution time (T_{1/2}) was directly proportional to the plasma coating time as shown in FIG. 2 and Table 2:

TABLE 2

| Plasma Deposition Time | T_{1/2} |
|---|---|
| (seconds) | (seconds) |
| 20 | 50 |
| 40 | 100 |
| 60 | 150 |
| 80 | 240 |

The plasma was generated at 83 W and 55 mTorr with a mass flow rate of 84 sccm; under these conditions a micro-thin siloxane membrane is deposited on the targeted surface, with the thickness directly related to the length of time of plasma deposition.

### EXAMPLE 3

Before drug application and plasma coating to 0.71 cm×0.71 cm 316 stainless steel coupon surfaces, all the coupons were cleaned with Acationox detergent, rinsed exhaustively with water, and air-dried. The coupons were then dip-coated in 80% ethanol containing 5% PHEMA and 5 mg/ml of daunomycin. After dipping, the coupons were wicked to remove excess solution, and air-dried under a gentle flow of warm air. Thereafter, the coated coupons were stored in the dark. The drug-coated coupons were then plasma-deposited with TMCTS for varying amounts of time. The plasma was generated at 83 W and 55 mTorr with a mass flow rate of 84 sccm of TMCTS monomer vapor. The coating thickness could be precisely controlled by plasma deposition time, with a coating thickness in the range of 5 to 200 nm.

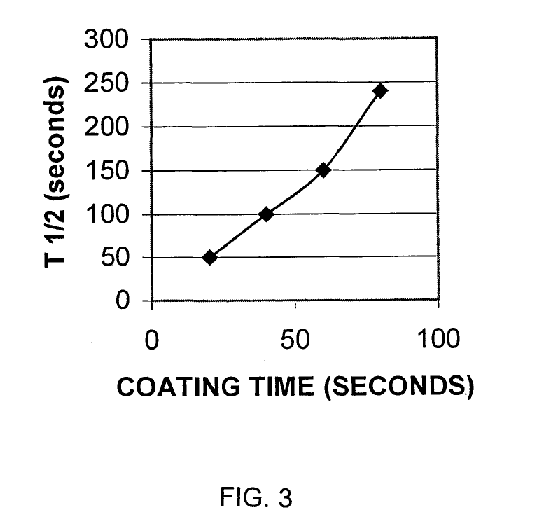

The coupons were eluted in buffered saline at pH 7.4. The eluted drug was measured spectrophotometrically at 480 nm for daunomycin. The absorbance change was proportional to the concentration of daunomycin. FIG. 3 shows the time course of the absorbance at 480 nm of the coupons with different plasma deposition times. The drug diffusion time was controlled by the TMCTS coating thickness.

### EXAMPLE 4

The elution time of rapamycin from the surface of stainless steel coupons was evaluated as a function of plasma deposition time. One side of stainless steel coupons was coated with 100±10 μg of rapamycin dissolved in chloroform (0.2%, w/v). After air drying at room temperature, the coated surfaces were plasma coated with TMCTS. The plasma was generated at 83 W and 55 mTorr with a mass flow rate of 84 sccm at various time lengths between 30 and 300 seconds. The coated coupons were extracted with porcine serum mixed with 0.02% NaN₃ as the preservative at 37° C. The remaining amounts of the drug on the coupon was extracted with 2 ml of methanol for 2 hours at room temperature and assayed by HPLC at 277 nm, utilizing an HPLC system calibrated with known amounts of rapamycin. The results are shown in Table 3.

TABLE 3

| | | | | |
|---|---|---|---|---|
| TMCTS Plasma | Elution | | | Estimated |
| Deposition Time | Time | Remained on | % | Half-Life |
| (Seconds) | (Hours) | Coupon (μg) | Remaining | (Hours) |

| | | | | |
|---|---|---|---|---|
| 0 (uncoated) | 0 | 91 ± 10 | 100 | |
| 0 (uncoated) | 17 | 0 | 0 | |
| 30 | 64 | 2.4 | 2.6 | 12 |
| 60 | 64 | 30.2 ± 6.8 | 33.2 | 41 |
| 120 | 47.5 | 61.6 ± 14.6 | 67.7 | 84 |
| 180 | 24 | 69.4 ± 15.6 | 76.3 | 94 |
| 240 | 24 | 85.2 ± 1.2 | 93.6 | 211 |
| 300 | 47.5 | 83.6 ± 4.2 | 91.9 | 360 |

The preceding examples can be repeated with similar success by substituting the generically or specifically described reactants and/or operating conditions of this invention for those used in the preceding examples.

Although the invention has been described in detail with particular reference to these preferred embodiments, other embodiments can achieve the same results: Variations and modifications of the present invention will be obvious to those skilled in the art and it is intended to cover in the appended claims all such modifications and equivalents. The entire disclosures of all references, applications, patents, and publications cited above are hereby incorporated by reference.

What is claimed is:

1. A biocompatible coating composition for therapeutic agent diffusion in vivo comprising:
a layer comprising a therapeutic agent dispersed in a polymeric matrix; and
a membrane posited over the layer, the membrane formed from the plasma polymerization of hydrocyclosiloxane monomer of the general formula: 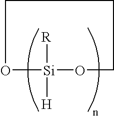
where R is an aliphatic group having 1 to about 5 carbon atoms and n is an integer from 2 to about 10, and
wherein the membrane cross-links with the polymeric matrix of the layer.

2. The coating composition of claim 1, wherein n is 7 to 10.

3. The coating composition of claim 1, wherein n is 4 to 6.

4. The coating composition of claim 1, wherein n is 2 to 3.

5. The coating composition of claim 1, wherein the hydrocyclosiloxane monomer is selected from the group consisting of 1,3,5,7-tetramethylhydrocyclotetrasiloxane, 1,3,5,7,9-pentamethylhydrocyclopentasiloxane, 1,3,5,7,9,11-hexamethylhydrocyclohexasiloxane, and a mixture of 1,3,5,7,9-pentamethylcyclopentasiloxane and 1,3,5,6,9,11-hexamethylcyclohexasiloxane monomers.

6. The coating composition of claim 1, wherein the polymer is selected from the group consisting of poly(2-hydroxyethyl methacrylate), polycaprolactone and cellulose acetate butyrate.

7. The coating composition of claim 1, wherein the membrane has a thickness of between about 10 nm and about 450 nm.

8. The coating composition of claim 7, wherein the membrane has a thickness of between about 20 and about 250 nm.

9. The coating composition of claim 1, wherein the polymeric matrix contains between about 0.01 mg and about 5.0 mg of therapeutic agent per cm².

10. The coating composition of claim 9, wherein the polymeric matrix contains between about 0.1 mg and about 0.5 mg of therapeutic agent per cm².

11. A biocompatible coating composition for therapeutic agent diffusion in vivo comprising:
a layer comprising a therapeutic agent; and
a membrane posited over the layer, the membrane formed from the plasma polymerization of hydrocyclosiloxane monomer of the general formula: 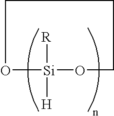
where R is an aliphatic group having 1 to about 5 carbon atoms and n is an integer from 2 to about 10, and
wherein the membrane cross-links with at least a portion of the therapeutic agent of the layer.

12. The coating composition of claim 11, wherein n is 7 to 10.

13. The coating composition of claim 11, wherein n is 4 to 6.

14. The coating composition of claim 11, wherein n is 2 to 3.

15. The coating composition of claim 11, wherein the hydrocyclosiloxane monomer is selected from the group consisting of 1,3,5,7-tetramethylhydrocyclotetrasiloxane, 1,3,5,7,9-pentamethylhydrocyclopentasiloxane, 1,3,5,7,9,1 1-hexamethylhydrocyclohexasiloxane, and a mixture of 1,3,5,7,9-pentamethylcyclopentasiloxane and 1,3,5,6,9,11-hexamethylcyclohexasiloxane monomers.

16. The coating composition of claim 11, wherein the membrane has a thickness of between about 10 nm and about 450 nm.

17. The coating composition of claim 16, wherein the membrane has a thickness of between about 20 and about 250 nm.

18. The coating composition of claim 1, wherein the layer comprising the therapeutic agent contains between about 0.01 mg and about 5.0 mg of therapeutic agent per cm².

19. The coating composition of claim 18, wherein the layer comprising the therapeutic agent contains between about 0.1 mg and about 0.5 mg of therapeutic agent per cm².

20. An implantable medical device comprising:
a structural component adapted for implantation in a patient, the structural component comprising at least one exterior surface;
a layer comprising a therapeutic agent posited over at least a portion of the at least one exterior surface; and
a membrane posited over the layer, the membrane formed from the plasma polymerization of hydrocyclosiloxane monomer of the general formula: 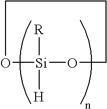
where R is an aliphatic group having 1 to about 5 carbon atoms and n is an integer from 2 to about 10.

21. The implantable medical device of claim 20, wherein the layer comprising a therapeutic agent further comprises a polymeric matrix, whereby the therapeutic agent is dispersed in the polymeric matrix and the membrane cross-links with the polymeric matrix.

22. The implantable medical device of claim 20, wherein n is 7 to 10.

23. The implantable medical device of claim 20, wherein n is 4 to 6.

24. The implantable medical device of claim 20, wherein n is 2 to 3.

25. The implantable medical device of claim 20, wherein the hydrocyclosiloxane monomer is selected from the group consisting of 1,3,5,7-tetramethylhydrocyclotetrasiloxane, 1,3,5,7,9-pentamethylhydrocyclopentasiloxane, 1,3,5,7,9,11-hexamethylhydrocyclohexasiloxane, and a mixture of 1,3,5,7,9-pentamethylcyclopentasiloxane and 1,3,5,6,9,11-hexamethylcyclohexasiloxane monomers.

26. The implantable medical device of claim 21, wherein the polymeric matrix comprises a polymer is selected from the group consisting of poly(2-hydroxyethyl methacrylate), polycaprolactone and cellulose acetate butyrate.

27. The implantable medical device of claim 20, wherein the membrane has a thickness of between about 10 nm and about 450 nm.

28. The implantable medical device of claim 20, wherein the membrane has a thickness of between about 20 and about 250 nm.

29. The implantable medical device of claim 20, wherein the layer comprising a therapeutic agent contains between about 0.01 mg and about 5.0 mg of therapeutic agent per cm².

30. The implantable medical device of claim 29, wherein the layer comprising a therapeutic agent contains between about 0.1 mg and about 0.5 mg of therapeutic agent per cm².

31. The implantable medical device of claim 20, wherein the structural component comprises a medical device selected from the group consisting of stents, catheters, shunts, grafts, artificial blood vessels, nerve-growth guides, artificial heart valves, joint prosthetics, pacemaker leads, cardiovascular grafts, bone replacements, orthopedic plates and attachments, wound healing devices, cartilage replacement devices and urinary tract replacement devices.

32. A method of applying a biocompatible coating composition to a structural component for therapeutic agent diffusion in vivo comprising:
providing a structural component adapted for introduction into a patient, the structural component comprising at least one exterior surface;
positing a layer comprising a therapeutic agent over at least a portion of the at least one exterior surface; and
plasma depositing a membrane over the layer comprising a therapeutic agent, the membrane formed from the plasma polymerization of hydrocyclosiloxane monomer of the general formula: 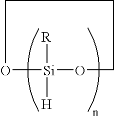
where R is an aliphatic group having 1 to about 5 carbon atoms and n is an integer from 2 to about 10.

33. The method of claim 32, wherein positing the layer comprising a therapeutic agent further comprises positing a layer comprising a polymeric matrix and the therapeutic agent.

34. The method of claim 32, wherein n is 7 to 10.

35. The method of claim 32, wherein n is 4 to 6.

36. The method of claim 32, wherein n is 2 to 3.

37. The method of claim 32, wherein the hydrocyclosiloxane monomer is selected from the group consisting of 1,3,5,7-tetramethylhydrocyclotetrasiloxane, 1,3,5,7,9-pentamethylhydrocyclopentasiloxane, 1,3,5,7,9,11-hexamethylhydrocyclohexasiloxane, and a mixture of 1,3,5,7,9-pentamethylcyclopentasiloxane and 1,3,5,6,9,11-hexamethylcyclohexasiloxane monomers.

38. The method of claim 33, wherein the polymeric matrix comprises a polymer is selected from the group consisting of poly(2-hydroxyethyl methacrylate), polycaprolactone and cellulose acetate butyrate.

39. The method of claim 32, wherein the membrane has a thickness of between about 10 nm and about 450 nm.

40. The method of claim 39, wherein the membrane has a thickness of between about 20 and about 250 nm.

41. The method of claim 32, wherein the layer comprising a therapeutic agent contains between about 0.01 mg and about 5.0 mg of therapeutic agent per cm².

42. The method of claim 41, wherein the layer comprising a therapeutic agent contains between about 0.1 mg and about 0.5 mg of therapeutic agent per cm².
